(19) United States
(12) Patent Application Publication
(10) Pub. No.: US 2025/0034184 A1
(43) Pub. Date: Jan. 30, 2025

(54) QUATERNARY PHOSPHONIUM COMPOUNDS AND USES THEREOF

(71) Applicants: Emory University, Atlanta, GA (US); Villanova University, Villanova, PA (US)

(72) Inventors: William Wuest, Atlanta, GA (US); Kevin Minbiole, Media, PA (US)

(73) Assignees: Emory University, Atlanta, GA (US); Villanova University, Villanova, PA (US)

(21) Appl. No.: 18/684,284

(22) PCT Filed: Aug. 17, 2022
(86) PCT No.: PCT/US2022/040541

§ 371 Date: Feb. 16, 2024

Related U.S. Application Data
(60) Provisional application No. 63/234,152, filed on Aug. 17, 2021.

(51) Int. Cl. C07F 9/54; A61K 31/66; A61P 31/04

(52) U.S. Cl. CPC C07F 9/5442; A61K 31/66; A61P 31/04; C07F 9/5456

(57) ABSTRACT

This disclosure relates to quaternary phosphonium compounds having lipophilic groups. In certain embodiments, this disclosure relates to quaternary phosphonium compounds having chemical formula as reported herein. In certain embodiments, this disclosure relates to compositions and devices comprising or coated with quaternary phosphonium compounds disclosed herein. In certain embodiments, this disclosure relates to preventing or treating a microbial infection or cancer using quaternary phosphonium compounds disclosed herein.

### CROSS-REFERENCE TO RELATED APPLICATIONS

This application claims the benefit of U.S. Provisional Application No. 63/234,152 filed Aug. 17, 2021. The entirety of this application is hereby incorporated by reference for all purposes.

### STATEMENT REGARDING FEDERALLY SPONSORED RESEARCH OR DEVELOPMENT

This invention was made with government support under GM119426 awarded by the National Institutes of Health. The government has certain rights in the invention.

### BACKGROUND

Quaternary ammonium compounds (QACs) are a staple antiseptic in household, agricultural, industrial, and clinical settings. Their popularity has continued to persist due to their broad-spectrum activity against a variety of microorganisms. While the non-specific mechanism of QACs suggested that these compounds would evade resistance development, unfortunately, tolerance was observed. QAC susceptibility were found to arise from alterations in membrane composition and stimulation of efflux systems encoded by the qac family of genes. Furthermore, sub-inhibitory concentrations of QAC treatments promote co-resistance via selection for the horizontal transfer of plasmids encoding both qac and other antimicrobial resistance genes. Thus, there is a need to identify improvements.

Kanazawa et al. report polymeric phosphonium salts as a class of cationic biocides. Journal of Polymer Science Part A: Polymer Chemistry, 1993, 31(12):3031-3038.

Kloepping et al. report triphenylphosphonium derivatives disrupt metabolism and inhibit melanoma growth in vivo when delivered via a thermosensitive hydrogel, PLoS ONE, 2020, 15(12): e0244540. See also U.S. Patent Publication No. 2015/0366884.

References cited herein are not an admission of prior art.

### SUMMARY

This disclosure relates to quaternary phosphonium compounds having lipophilic groups. In certain embodiments, this disclosure relates to quaternary phosphonium compounds having chemical formula as reported herein. In certain embodiments, this disclosure relates to compositions and devices comprising or coated with quaternary phosphonium compounds disclosed herein. In certain embodiments, this disclosure relates to preventing or treating a microbial infection or cancer using quaternary phosphonium compounds disclosed herein.

In certain embodiments, this disclosure relates to compositions and devices comprising a quaternary phosphonium compound disclosed herein. In certain embodiments, this disclosure relates to soaps and disinfectant products, pharmaceutical formulations, or medical devices comprising a quaternary phosphonium compound disclosed herein.

In certain embodiments, this disclosure relates to methods of treating or preventing diseases or conditions comprising administering an effective amount of a composition comprising a quaternary phosphonium compound disclosed herein to a subject in need thereof.

In certain embodiments, this disclosure relates to methods of treating or preventing a microbial infection comprising administering to a subject in need thereof an effective amount of a quaternary phosphonium compound as disclosed herein.

In certain embodiments, the microbial infection is a bacterial, fungal, pest, or viral infection.

In certain embodiments, this disclosure relates to methods of treating cancer using compounds disclosed herein.

In certain embodiments, this disclosure relates to the production of a medicament for therapeutic uses reported herein.

### BRIEF DESCRIPTION OF THE SEVERAL VIEWS OF THE DRAWINGS

FIG. 1 illustrates embodiments of this disclosure.

FIG. 2 shows a comparative bioinformatic analysis of the bacterial panel resistomes. Representative resistance-mediating efflux pumps from the five multidrug efflux pump families are shown, highlighting the unique presence of qacC, qacE, and emrE in strains refractory to QAC and QPC treatments. (MFS=Major Facilitator Superfamily; RND=Resistance/Nodulation/cell Division family; ABC=ATP-Binding Cassette family; MATE=Multidrug and Toxic compound Extrusion family; SMR=Small Multidrug Resistance family).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature. Prior to describing the various embodiments, the following definitions are provided and should be used unless otherwise indicated.

An “embodiment” of this disclosure refers to an example and infers that the example is not necessarily limited to the example.

As used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), have the meaning ascribed to them in U.S. Patent law in that they are inclusive or open-ended and do not exclude additional, unrecited elements or method steps. “Consisting essentially of” or “consists essentially” or the like, when applied to methods and compositions encompassed by the present disclosure refers to compositions like those disclosed herein that exclude certain prior art elements to provide an inventive feature of a claim, but which may contain additional composition components or method steps composition components or method steps, etc., that do not materially affect the basic and novel characteristic(s) of the compositions or methods, compared to those of the corresponding compositions or methods disclosed herein.

The term “microorganism” or “microbe” as used herein refers to a small (often, but not always, microscopic) organism that is typically, but not exclusively, single cellular, and includes organisms from the kingdoms bacteria, archaea, protozoa, and fungi. The present disclosure is primarily directed to microorganisms that are pathogenic and capable of causing disease. Such microbes may be detected from a sample. In embodiments, microorganism includes bacteria and fungi capable of causing disease, particularly disease in humans and other mammals and animals in need of treatment.

A “subject” refers any animal, preferably a human patient, livestock, rodent, monkey, or domestic pet. In certain embodiments, the subject may be exhibiting symptoms of, at risk of, or diagnosed with a disease or condition by analysis of a sample. The term “sample” can refer to a tissue sample, cell sample, a fluid sample, and the like. A sample may be taken from a host subject. The tissue sample can include hair, buccal swabs, blood, saliva, semen, muscle, or tissue from any internal organ. The fluid may be, but is not limited to, urine, blood, ascites, pleural fluid, spinal fluid, semen, wound exudates, sputum, fecal matter, saliva, and the like. The body tissue can include, but is not limited to, skin, muscle, endometrial, uterine, and cervical tissue. While a sample, in the context of the present disclosure, is primarily a biological sample (e.g., from a living host) the sample may also be an environmental sample suspected of contamination by microbes, such as a water sample, food sample, soil sample, and the like. Although a liquid sample and some solid samples may be used as a test sample without modification for testing directly, if a solid sample is to be made into liquid form for testing and/or a liquid sample is to be diluted, a test sample may be made by reconstituting, dissolving, or diluting the sample in a fluid such as water, buffered saline, and the like.

As used herein, the terms “prevent” and “preventing” include the prevention of the recurrence, spread or onset. It is not intended that the present disclosure be limited to complete prevention. In some embodiments, the onset is delayed, or the severity is reduced.

As used herein, the terms “treat” and “treating” are not limited to the case where the subject (e.g. patient) is cured and the disease is eradicated. Rather, embodiments of the present disclosure also contemplate treatment that merely reduces symptoms, and/or delays disease progression.

The term “effective amount” or “therapeutically effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on, for example, the particular compounds chosen, the dosing regimen to be followed, whether it is administered in combination with other agents, timing of administration, the tissue to which it is administered, and the physical delivery system in which it is carried.

As used herein, a “lipid” group refers to a hydrophobic group that is naturally or non-naturally occurring and is highly insoluble in water. As used herein a substituent lipid group is considered highly insoluble in water when the point of connection on the lipid is replaced with a hydrogen and the resulting compound has a solubility of less than 0.63×10-4% w/w (at 25° C.) in water, which is the percent solubility of octane in water by weight. See Solvent Recovery Handbook, 2nd Ed, Smallwood, 2002 by Blackwell Science, page 195. Examples of naturally occurring lipids include saturated or unsaturated hydrocarbon chains found in fatty acids, glycerolipids, cholesterol, steroids, polyketides, and derivatives. Non-naturally occurring lipids include derivatives of naturally occurring lipids, acrylic polymers, aromatic, and alkylated compounds and derivatives thereof.

A “linking group” refers to any variety of molecular arrangements that can be used to bridge molecular moieties together. An example formula may be —Rₙ— wherein R is selected individually and independently at each occurrence as: —CRₙRₙ—, —CHRₙ—, —CH—, —C—, —CH₂—, —C(OH)Rₙ, —C(OH)(OH)—, —C(OH)H, —C(Hal)Rₙ—, —C(Hal)(Hal)-, —C(Hal)H—, —C(N₃)Rₙ—, —C(CN)Rₙ—, —C(CN)(CN)—, —C(CN)H—, —C(N₃)(N₃)—, —C(N₃)H—, —O—, —S—, —N—, —NH—, —NRₙ—, —(C═O)—, —(C═NH)—, —(C═S)—, —(C═CH₂)—, which may contain single, double, or triple bonds individually and independently between the R groups. If an R is branched with an Rₙ it may be terminated with a group such as —CH₃, —H, —CH═CH₂, —CCH, —OH, —SH, —NH₂, —N₃, —CN, or -Hal, or two branched Rs may form a cyclic structure. It is contemplated that in certain instances, the total Rs or “n” may be less than 100 or 50 or 25 or 10. Examples of linking groups include bridging alkyl groups and alkoxyalkyl groups.

In certain embodiments, this disclosure contemplates derivatives of compounds disclosed herein. As used herein, the term “derivative” refers to a structurally similar compound that retains sufficient functional attributes of the identified analogue. The derivative may be structurally similar because it is lacking one or more atoms, substituted, a salt, or alternative salt, in different hydration/oxidation states, or because one or more atoms within the molecule are switched, such as, but not limited to, replacing an oxygen atom with a sulfur atom, or replacing an amino group with a hydroxy group. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze, hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxy, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

As used herein, “alkyl” means a noncyclic straight chain or branched, unsaturated or saturated hydrocarbon such as those containing from 1 to 10 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

The terms, “cell culture” or “growth medium” or “media” refers to a composition that contains components that facilitate cell maintenance and growth through protein biosynthesis, such as vitamins, amino acids, inorganic salts, a buffer, and a fuel, e.g., acetate, succinate, a saccharide/disaccharide/polysaccharide, medium chain fatty acids, and/or optionally nucleotides. Typical components in a growth medium include amino acids (histidine, isoleucine, leucine, lysine, methionine, phenylalanine, threonine, tryptophan, valine and others); vitamins such as retinol, carotene, thiamine, riboflavin, niacin, biotin, folate, and ascorbic acid; carbohydrate such as glucose, galactose, fructose, or maltose; inorganic salts such as sodium, calcium, iron, potassium, magnesium, zinc; serum; and buffering agents. Additionally, a growth medium may contain phenol red as a pH indication.

### Quaternary Phosphonium Compounds

Quaternary phosphonium compounds (QPCs) are characterized by their tetravalent phosphorous atom with a positive formal charge. This disclosure relates to quaternary phosphonium compounds having lipophilic groups. As quaternary phosphonium is a cationic salt, such “compounds” include composition comprising any variety of counter anions.

In certain embodiments, this disclosure relates to quaternary phosphonium compounds having chemical formula as reported herein. In certain embodiments, the quaternary phosphonium compound has Formula I,

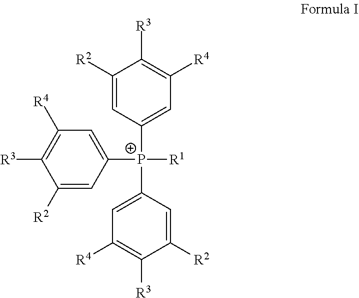

wherein
R¹ is a lipid and
R², R³, and R⁴ are each individually and independently selected from hydrogen, alkyl, alkoxy, or halogen, wherein R², R³, and R⁴ are optionally substituted.

In certain embodiments, the R¹ lipid is a saturated or unsaturated hydrocarbon with eight or greater carbons.

In certain embodiments, quaternary phosphonium compound is selected from:
octyltriphenylphosphonium,
decyltriphenylphosphonium,
triphenyl(undecyl)phosphonium,
dodecyltriphenylphosphonium,
triphenyl(tridecyl)phosphonium,
triphenyl(tetradecyl)phosphonium,
hexadecyltriphenylphosphonium, and
octadecyltriphenylphosphonium.

In certain embodiments, the quaternary phosphonium compound has Formula II,

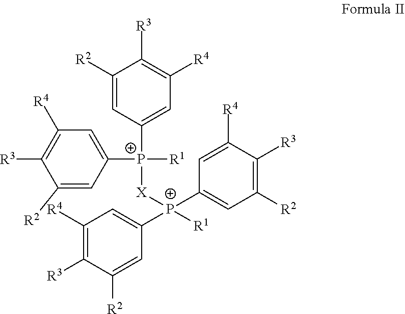

wherein,
R¹ is a lipid;
X is a linking group; and
R², R³, and R⁴ are each individually and independently selected from hydrogen, alkyl, alkoxy, or halogen, wherein R², R³, and R⁴ are optionally substituted.

In certain embodiments, the R¹ lipid is a saturated or unsaturated hydrocarbon with eight or greater carbons.

In certain embodiments, the X linking group is a saturated or unsaturated hydrocarbon with two or greater carbons.

In certain embodiments, quaternary phosphonium compound is selected from:
ethane-1,2-diylbis(octyldiphenylphosphonium),
ethane-1,2-diylbis(decyldiphenylphosphonium),
ethane-1,2-diylbis(undecyldiphenylphosphonium),
ethane-1,2-diylbis(dodecyldiphenylphosphonium),
ethane-1,2-diylbis(tridecyldiphenylphosphonium),
ethane-1,2-diylbis(tetradecyldiphenylphosphonium),
ethane-1,2-diylbis(hexadecyldiphenylphosphonium),
ethane-1,2-diylbis(octadecyldiphenylphosphonium),
propane-1,3-diylbis(octyldiphenylphosphonium),
propane-1,3-diylbis(decyldiphenylphosphonium),
propane-1,3-diylbis(undecyldiphenylphosphonium),
propane-1,3-diylbis(dodecyldiphenylphosphonium),
propane-1,3-diylbis(tridecyldiphenylphosphonium),
propane-1,3-diylbis(tetradecyldiphenylphosphonium),
propane-1,3-diylbis(hexadecyldiphenylphosphonium),
propane-1,3-diylbis(octadecyldiphenylphosphonium),
butane-1,4-diylbis(octyldiphenylphosphonium),
butane-1,4-diylbis(decyldiphenylphosphonium),
butane-1,4-diylbis(undecyldiphenylphosphonium),
butane-1,4-diylbis(dodecyldiphenylphosphonium),
butane-1,4-diylbis(tridecyldiphenylphosphonium),
butane-1,4-diylbis(tetradecyldiphenylphosphonium),
butane-1,4-diylbis(hexadecyldiphenylphosphonium),
butane-1,4-diylbis(octadecyldiphenylphosphonium),
pentane-1,5-diylbis(octyldiphenylphosphonium),
pentane-1,5-diylbis(decyldiphenylphosphonium),
pentane-1,5-diylbis(undecyldiphenylphosphonium),
pentane-1,5-diylbis(dodecyldiphenylphosphonium),
pentane-1,5-diylbis(tridecyldiphenylphosphonium),
pentane-1,5-diylbis(tetradecyldiphenylphosphonium),
pentane-1,5-diylbis(hexadecyldiphenylphosphonium),
pentane-1,5-diylbis(octadecyldiphenylphosphonium),
hexane-1,6-diylbis(octyldiphenylphosphonium),
hexane-1,6-diylbis(decyldiphenylphosphonium),
hexane-1,6-diylbis(undecyldiphenylphosphonium),
hexane-1,6-diylbis(dodecyldiphenylphosphonium),
hexane-1,6-diylbis(tridecyldiphenylphosphonium),
hexane-1,6-diylbis(tetradecyldiphenylphosphonium),
hexane-1,6-diylbis(hexadecyldiphenylphosphonium), and
hexane-1,6-diylbis(octadecyldiphenylphosphonium).

In certain embodiments, the quaternary phosphonium compound has Formula III,

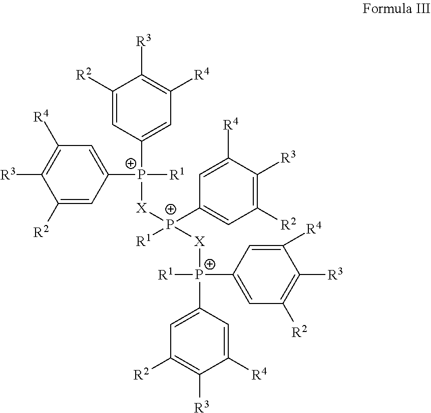

wherein,
R¹ is a lipid;
X is a linking group; and
R², R³, and R⁴ are each individually and independently selected from hydrogen, alkyl, alkoxy, or halogen, wherein R², R³, and R⁴ are optionally substituted.

In certain embodiments, the R¹ lipid is a saturated or unsaturated hydrocarbon with eight or greater carbons.

In certain embodiments, the X linking group is a saturated or unsaturated hydrocarbon with two or greater carbons.

In certain embodiments, quaternary phosphonium compound is selected from:
((octyl(phenyl)phosphoniodiyl)bis(ethane-2,1-diyl))bis(octyldiphenylphosphonium),
((decyl(phenyl)phosphoniodiyl)bis(ethane-2,1-diyl))bis(decyldiphenylphosphonium),
((phenyl(undecyl)phosphoniodiyl)bis(ethane-2,1-diyl))bis(diphenyl(undecyl)phosphonium),
((dodecyl(phenyl)phosphoniodiyl)bis(ethane-2,1-diyl))bis(dodecyldiphenylphosphonium),
((phenyl(tridecyl)phosphoniodiyl)bis(ethane-2,1-diyl))bis(diphenyl(tridecyl)phosphonium),
((phenyl(tetradecyl)phosphoniodiyl)bis(ethane-2,1-diyl))bis(diphenyl(tetradecyl)phosphonium),
((hexadecyl(phenyl)phosphoniodiyl)bis(ethane-2,1-diyl))bis(hexadecyldiphenylphosphonium), and
((octadecyl(phenyl)phosphoniodiyl)bis(ethane-2,1-diyl))bis(octadecyldiphenylphosphonium).

In certain embodiments, the quaternary phosphonium compound has Formula IV,

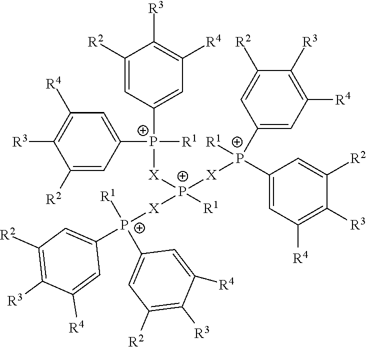

wherein,
R¹ is a lipid;
X is a linking group; and
R², R³, and R⁴ are each individually and independently selected from hydrogen, alkyl, alkoxy, or halogen, wherein R², R³, and R⁴ are optionally substituted.

In certain embodiments, the R¹ lipid is a saturated or unsaturated hydrocarbon with eight or greater carbons.

In certain embodiments, the X linking group is a saturated or unsaturated hydrocarbon with two or greater carbons.

In certain embodiments, quaternary phosphonium compound is selected from:
((decylphosphoniotriyl)tris(ethane-2,1-diyl))tris(decyldiphenylphosphonium),
((undecylphosphoniotriyl)tris(ethane-2,1-diyl))tris(diphenyl(undecyl)phosphonium), and
((dodecylphosphoniotriyl)tris(ethane-2,1-diyl))tris(dodecyldiphenylphosphonium).

### Methods of Use

In certain embodiments, this disclosure relates to methods of treating or preventing diseases or conditions comprising administering an effective amount of a composition comprising a quaternary phosphonium compound disclosed herein to a subject in need thereof.

In certain embodiments, the disclosure contemplates that a quaternary phosphonium compound disclosed herein may be used in antimicrobial applications optionally in combination with other antimicrobial agents for prevention of disease onset and treatment. In certain embodiments, the quaternary phosphonium compound disclosed herein may be used in medical device coatings (medical implants and tools, IV catheters), wound dressings (embedded in gauze bandages), wound rinses (i.e. surgical rinses), wound-vacuum systems, whole body baths (e.g., in combo with bleach baths for treatment of skin flares for atopic dermatitis/eczema), soaps, personal care products (body washes, lotions, soaps) for high risk patients or for populations with high risk of exposure (e.g. athletes using common sports equipment in gym), and veterinary applications (e.g. anti-infectives for companion animals, race horses, etc.)

In certain embodiments, this disclosure relates to methods of treating or preventing a microbial infection comprising administering to a subject in need thereof an effective amount of a quaternary phosphonium compound as disclosed herein. In certain embodiments, the microbial infection is a bacterial, fungal, pest, or viral infection.

In certain embodiments, this disclosure relates to methods of treating or preventing bacterial infections comprising administering or contacting the skin of a subject with formula comprising a quaternary phosphonium compound as disclosed herein to a subject in need thereof. In certain embodiments, the formula is administered in combination with another antibiotic agent.

In certain embodiments, this disclosure relates to using quaternary phosphonium compounds disclosed herein for treating or preventing an Acinetobacter baumannii infection, other bacterial infection, other multidrug resistant bacteria, or other microbial infection by administering an effective amount of quaternary phosphonium compounds disclosed herein to a subject in need thereof. In further embodiments, the quaternary phosphonium compound is co-administered with an antibiotic selected from the group comprising of sulfonamides, diaminopyrimidines, quinolones, beta-lactam antibiotics, cephalosporins, tetracyclines, nitrobenzene derivatives, aminoglycosides, macrolide antibiotics, polypeptide antibiotics, nitrofuran derivatives, nitroimidazoles, nicotinic acid derivatives, polyene antibiotics, imidazole derivatives or glycopeptide, cyclic lipopeptides, glycylcyclines and oxazolidinones. In further embodiments, these antibiotics include but are not limited to sulphadiazine, sulphones—[dapsone (DDS) and para-aminosalicylic (PAS)], sulfanilamide, sulfamethizole, sulfamethoxazole, sulphapyridine, trimethoprim, pyrimethamine, nalidixic acids, norfloxacin, ciprofloxacin, cinoxacin, enoxacin, gatifloxacin, gemifloxacin, grepafloxacin, levofloxacin, lomefloxacin, moxifloxacin, ofloxacin, pefloxacin, sparfloxacin, trovafloxacin, penicillins (amoxicillin, ampicillin, azlocillin, carbenicillin, cloxacillin, dicloxacillin, flucloxacillin, hetacillin, oxacillin, mezlocillin, penicillin G, penicillin V, piperacillin), cephalosporins (cefacetrile, cefadroxil, cefalexin, cephaloglycin, cefalonium, cefaloridine, cefalotin, cefapirin, cefatrizine, cefazaflur, cefazedone, cefazolin, cefradine, cefroxadine, ceftezole, cefaclor, cefonicid, ceforanide, cefprozil, cefuroxime, cefuzonam, cefmetazole, cefotetan, cefoxitin, cefcapene, cefdaloxime, cefdinir, cefditoren, cefetamet, cefixime, cefmenoxime, cefodizime, cefoperazone, cefotaxime, cefotiam, cefpimizole, cefpiramide, cefpodoxime, cefteram, ceftibuten, ceftiofur, ceftizoxime, ceftriaxone, cefoperazone, ceftazidime, cefepime), moxalactam, carbapenems (imipenem, ertapenem, meropenem) monobactams (aztreonam), oxytetracycline, chlortetracycline, clomocycline, demeclocycline, tetracycline, doxycycline, lymecycline, meclocycline, methacycline, minocycline, rolitetracycline, chloramphenicol, amikacin, gentamicin, framycetin, kanamycin, neomycin, netilmicin, streptomycin, tobramycin, azithromycin, clarithromycin, dirithromycin, erythromycin, roxithromycin, telithromycin, colistin, bacitracin, tyrothricin, nitrofurantoin, furazolidone, metronidazole, tinidazole, isoniazid, pyrazinamide, ethionamide, nystatin, amphotericin-B, hamycin, miconazole, clotrimazole, ketoconazole, fluconazole, rifampicin, lincomycin, clindamycin, spectinomycin, fosfomycin, loracarbef, polymyxin B, polymyxin B Sulphate, procaine, ramoplanin, teicoplanin, vancomycin, and/or nitrofurantoin.

In certain embodiments, the subject is diagnosed with a bacterial infection. In certain embodiments, the subject is diagnosed with bacteremia, pneumonia, staphylococcal food poisoning, necrotizing pneumonia, necrotizing fasciitis, scalded skin syndrome, post-operation bacterial infection, medical device bacterial infection, bacterial infection of the skin, soft tissue bacterial infection, or toxic shock syndrome.

In certain embodiments, this disclosure relates to methods of treating or preventing a toxin-mediated bacterial infection comprising administering an effective amount of a quaternary phosphonium compound as disclosed herein to a subject in need thereof, including a subject at risk of, exhibiting symptoms of, or diagnosed with scalded skin syndrome (esp. in neonates), abscesses, necrotizing fasciitis, sepsis, or atopic dermatitis (eczema).

In certain embodiments, this disclosure relates to methods of treating or preventing bacterial infections or acne comprising administering to a subject in need thereof or contacting the skin of a subject in need thereof with a formula comprising of a quaternary phosphonium compound as disclosed herein. In certain embodiments, the formula is administered in combination with another antibiotic.

In certain embodiments, the subject is at risk of a bacterial infection due to being diagnosed with an abscess, furuncle, cellulitis, folliculitis, atopic dermatitis, psoriasis, impetigo, septic arthritis, brain abscess, burn wound, venous ulcer, diabetic foot ulcer, surgical wound, carbuncle, or meningitis.

A flow of wound rinse/irrigation solution can be applied across an open wound surface to achieve wound hydration, to remove deeper debris, and to assist with the visual examination. In certain embodiments, the disclosure relates to methods of irrigating a wound using a solution comprising a quaternary phosphonium compound as disclosed herein.

In certain embodiments, this disclosure relates to using quaternary phosphonium compounds disclosed herein for killing microbes, preventing a biofilm formation, or preventing the spread of an Acinetobacter baumannii infection, other bacterial infection, other multidrug resistant bacteria, or other microbial infection by sanitizing a surface, e.g., by contacting the surface with a solid (e.g., powder), liquid, or spray composition, with a quaternary phosphonium compound disclosed herein in an effective amount.

In certain embodiments, this disclosure relates to methods of treating cancer using compounds disclosed herein. “Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

The cancer to be treated in the context of the present disclosure may be any type of cancer or tumor.

In certain embodiments, this disclosure relates to methods of treating or preventing melanoma comprising administering to a subject in need thereof an effective amount of a quaternary phosphonium compound as disclosed herein.

Also contemplated are malignancies located in the brain (glioma, glioblastoma), colon, abdomen, bone, breast, digestive system, liver, pancreas, kidney, lung, skin, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, or thorax.

In certain embodiments, the cancer may be a hematological malignancy such as leukemia, lymphoma, or myeloma.

In certain embodiments, this disclosure relates to methods of preventing cellular infections comprising applying a quaternary phosphonium compound disclosed herein on top of or inside a cell growth medium.

In certain embodiments, this disclosure relates to methods of preventing plant microbial infections comprising applying a quaternary phosphonium compound disclosed herein to the exterior, leaf, seed, or stem of a plant. In certain embodiments, this disclosure relates to methods of preventing plant microbial infections comprising applying a quaternary phosphonium compound disclosed herein on top of or into soil, dirt, sand, or other medium from which roots of the plant reside.

### Compositions and Devices

In certain embodiments, this disclosure relates to compositions and devices comprising a quaternary phosphonium compound disclosed herein.

In certain embodiments, this disclosure relates to soaps and disinfectant products comprising a quaternary phosphonium compound disclosed herein. Contemplated topical formulations for skin flares (i.e., for atopic dermatitis or other infections related to a disrupted skin barrier) may be combined with another drug such as a topical steroid, anti-inflammatory agent, and promoter of skin barrier function or skin moisturizer. In certain embodiments, this disclosure relates a container configured to create a liquid spray comprising a quaternary phosphonium compound disclosed herein.

In certain embodiments, this disclosure relates to pharmaceutical formulation comprising a quaternary phosphonium compound disclosed herein and a pharmaceutically acceptable excipient. In certain embodiments, the pharmaceutical formulation is in the form of a lotion, liquid, or gel. In certain embodiment, the pharmaceutical formulation is in the form of a particle, bead, tablet, capsule, pill, or injectable solution. The injectable solutions or suspensions may be formulated according to known art, using suitable non-toxic, parenterally-acceptable diluents or solvents, such as water, dimethyl sulfoxide, mannitol, 1,3-butanediol, Ringer's solution or isotonic sodium chloride solution, or suitable dispersing or wetting and suspending agents, such as sterile, bland, fixed oils, including synthetic mono- or diglycerides, and fatty acids, including oleic acid.

The pharmaceutical formulation can also include any type of pharmaceutically acceptable excipients, additives, or vehicles. For example, diluents or fillers, such as dextrates, dicalcium phosphate, calcium sulphate, lactose, cellulose, kaolin, mannitol, sodium chloride, dry starch, sorbitol, sucrose, inositol, powdered sugar, bentonite, microcrystalline cellulose, or hydroxypropyl methylcellulose, may be added to the composition to increase the bulk of the composition.

In certain embodiments, the formulation is a directly compressible composition comprising a quaternary phosphonium compound disclosed herein but no excipients, additives, or vehicles.

In certain embodiments, the disclosure relates to a pharmaceutical or cosmetic formulation comprising a quaternary phosphonium compound disclosed herein and a pharmaceutically acceptable excipient or cosmetically acceptable excipient. In certain embodiments, the disclosure relates to a liquid or gel formulation optionally further comprising an antibacterial agent, a topical steroid, an anti-inflammatory agent, a promoter of skin barrier function, a skin moisturizer, or combinations thereof. In certain embodiments the antibacterial agent is daptomycin, linezolid, vancomycin, nafcillin, cefazolin, dicloxacillin, clindamycin, rifampin, or sulfamethoxazole-trimethoprim (Bactrim).

In certain embodiments, the disclosure relates to a wound dressings or wound rinse comprising a quaternary phosphonium compound disclosed herein wherein the wound dressing comprises an absorbent pad and optionally an adhesive.

In certain embodiments, the disclosure relates to disinfectant sprays or wipes formulation for surfaces and fomites comprising a quaternary phosphonium compound disclosed herein.

In certain embodiments, this disclosure relates to medical device coated with a quaternary phosphonium compound as disclosed herein. In certain embodiments, the medical device is a screw, pin, plate, rod, disk, needle, catheter, tube, stent, pacemaker, defibrillators (ICDs), artificial hip or knee joint/implant, breast implant, intra-uterine device, ear tube, contact lens, or implantable pump.

In certain embodiments, this disclosure relates to surgical tools coated with a quaternary phosphonium compound as disclosed herein. In certain embodiments, the surgical tool is a forceps, tweezers, scalpel, knife, scissors, retractor, needle, gauze, sponge, suction, staple, stapler, clip, laparoscopic instrument, electrosurgical cauterizer, ultrasonic device, camera, camera lens, fiber optic cable, insufflator, needle, bronchoscope, cystoscope, saw, or robotic arm.

In certain embodiments, the disclosure relates to a wound dressing comprising a quaternary phosphonium compound as disclosed herein wherein the wound dress comprises an absorbent pad and optionally an adhesive optionally in combination with another antibiotic agent. In certain embodiments, the wound dressing is a foam or compression dressing or a cover dressing such as wraps, gauze and tape. In certain embodiments, the wound dressing comprises alginate or collagen. In certain embodiments, the wound dressing is a hydrocolloid dressing, e.g., carboxy-methylcellulose and gelatin optionally in a polyurethane foam or film, optionally comprising one or more agents selected from pectin, a polysaccharide, and an adhesive.

In certain embodiments, the wound dressing is a hydrogel. Hydrogels are polymers that contain a high content of hydroxy and/or carboxyl containing monomers or salts thereof, e.g., vinyl alcohol, acrylic acid, 2-hydroxyethylmethacrylate, ethylene glycol dimethacrylate monomers, which are co-polymers to provide varying degrees of hydration. Due to the hydrophilic monomers, the hydrogels typically absorb water. Contemplated hydrogel dressings include: amorphous hydrogel, which are a free-flowing gel that are typically distributed in tubes, foil packets and spray bottles; an impregnated hydrogel, which are typically saturated onto a gauze pad, nonwoven sponge ropes and/or strips; or a sheet hydrogel which are gel held together by a fiber mesh.

In certain embodiments, the disclosure relates to a wound rinse comprising a quaternary phosphonium compound as disclosed herein optionally containing normal saline, sterile water, a detergent, a surfactant, a preservative, or iodine.

In certain embodiments, the disclosure contemplates a kit comprising a container comprising a quaternary phosphonium compound as disclosed herein optionally comprising a second container comprising a rinse solution or containing surgical device or tool, normal saline, sterile water, a detergent, a surfactant, a preservative, iodine, hydrogen peroxide, or sodium hypochlorite or other compound disclosed herein.

In certain embodiments, the disclosure relates to a cosmetic formulation comprising a quaternary phosphonium compound as disclosed herein and cosmetically acceptable excipient or additive. In certain embodiments, the disclosure relates to a solid or liquid soap or lotion comprising a quaternary phosphonium compound as disclosed herein and a fatty acid. In certain embodiments, additives can be selected from the group consisting of oily bodies, surfactants, emulsifiers, fats, waxes, pearlescent waxes, bodying agents, thickeners, superfatting agents, stabilizers, polymers, silicone compounds, lecithins, phospholipids, biogenic active ingredients, deodorants, antimicrobial agents, antiperspirants, film formers, antidandruff agents, swelling agents, insect repellents, hydrotropes, solubilizers, preservatives, perfume oils and dyes. In certain embodiments, additives are selected from the group consisting of surfactants, emulsifiers, fats, waxes, stabilizers, deodorants, antiperspirants, antidandruff agents, and perfume oils.

In certain embodiments, this disclosure relates to a cell growth medium comprising a quaternary phosphonium compound disclosed herein.

### EXAMPLES

### Preparation of Quaternary Phosphonium Compounds.

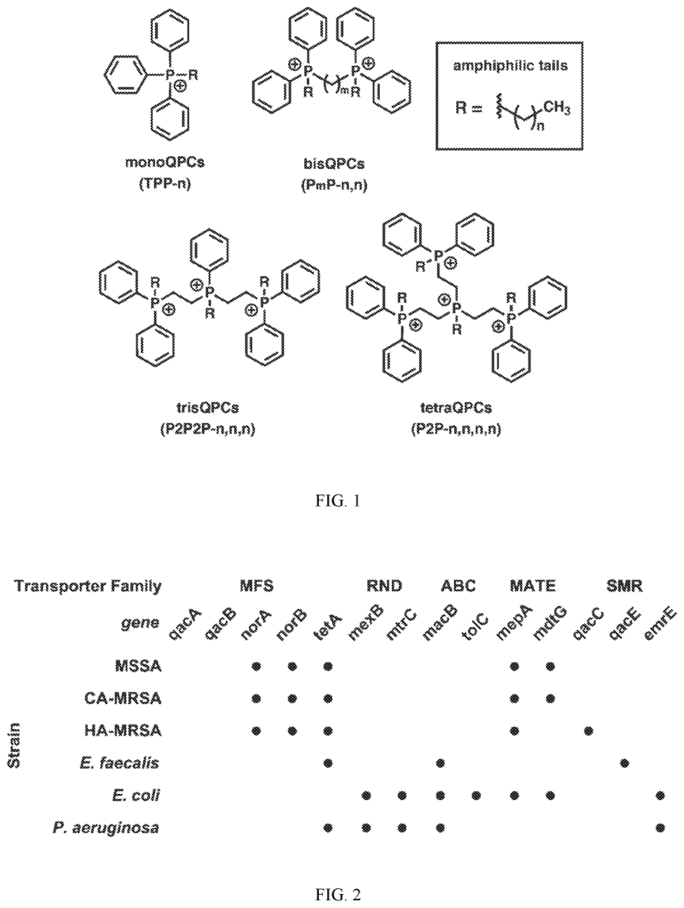

It was contemplated that alkylation may be achieved through quaternization of phenylphosphine compounds such as 1,3-bis(diphenylphosphino) propane (dppp) and triphenylphosphine (TPP) due to their relative air stabilities. Experiments with the synthesis of TPP- and dppp-derived QPCs bearing hydrocarbon tails of varying lengths were performed (FIG. 1) in order to determine whether increasing the amphiphilic and cationic nature of the QPCs may increase broad-spectrum antimicrobial activity and aid in the evasion of efflux-pump mediated resistance. Experiments were performed to determine whether access to monoQPCs could be gained via alkylation of TPP. In an initial experiment, TPP was treated with 1.5 equivalents of 1-bromotetradecance in acetonitrile at reflux for 72 h, which afforded TPP-14 in 73% yield (Table 1).

TABLE 1
Preparation of monoQPCs.

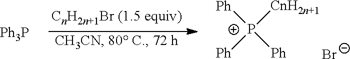

| compd. | name | n | yield (%) |
|---|---|---|---|
| 1 | TPP-8 | 8 | 75 |
| 2 | TPP-10 | 10 | 93 |
| 3 | TPP-11 | 11 | 96 |
| 4 | TPP-12 | 12 | 96 |
| 5 | TPP-13 | 13 | 97 |
| 6 | TPP-14 | 14 | 73 |
| 7 | TPP-16 | 16 | 68 |
| 8 | TPP-18 | 18 | 77 |

These conditions were found to be broadly effective for the synthesis of monoQPCs bearing hydrocarbon tails of 8-18 carbons in length. MonoQPCs were prepared in yields ranging from 68-97% and labeled TPP-n, in order to represent the triphenylphosphine core and respective tail length (n).

To examine phenylphosphonium compounds containing multiple sites for quaternization, methods of synthesizing bisQPCs were investigated. Members from this QPC subclass were derived from alkyl linked diphenylphosphine moieties. Beginning with dppp, an initial experiment with excess 1-bromohexadecane (2.5 equivalents) in acetonitrile at reflux for 48 h produced the corresponding bis-cationic phosphonium salt in 85% yield (Table 2). This method was then applied to the generation of seven additional dppp-derived bisQPCs.

TABLE 2
Preparation of bisQPCs

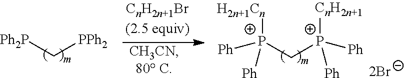

| compd. | name | m | n | timed (h) | yield (%) |
|---|---|---|---|---|---|
| 9 | P2P-8,8 | 2 | 8 | 96 | 91 |
| 10 | P2P-10,10 | 2 | 10 | 96 | 94 |
| 11 | P2P-11,11 | 2 | 11 | 96 | 89 |
| 12 | P2P-12,12 | 2 | 12 | 96 | 95 |
| 13 | P2P-13,13 | 2 | 13 | 96 | 92 |
| 14 | P2P-14,14 | 2 | 14 | 96 | 93 |
| 15 | P2P-16,16 | 2 | 16 | 96 | 93 |
| 16 | P2P-18,18 | 2 | 18 | 96 | 86 |
| 17 | P3P-8,8 | 3 | 8 | 25 | 85 |
| 18 | P3P-10,10 | 3 | 10 | 24 | 90 |
| 19 | P3P-11,11 | 3 | 11 | 25 | 90 |
| 20 | P3P-12,12 | 3 | 12 | 27 | 90 |
| 21 | P3P-13,13 | 3 | 13 | 26 | 90 |
| 22 | P3P-14,14 | 3 | 14 | 26 | 91 |
| 23 | P3P-16,16 | 3 | 16 | 25 | 85 |
| 24 | P3P-18,18 | 3 | 18 | 48 | 78 |
| 25 | P4P-8,8 | 4 | 8 | 26 | >99 |
| 26 | P4P-10,10 | 4 | 10 | 26 | 96 |
| 27 | P4P-11,11 | 4 | 11 | 24 | 90 |
| 28 | P4P-12,12 | 4 | 12 | 27 | 91 |
| 29 | P4P-13,13 | 4 | 13 | 28 | 91 |
| 30 | P4P-14,14 | 4 | 14 | 28 | 89 |
| 31 | P4P-16,16 | 4 | 16 | 28 | 81 |
| 32 | P4P-18,18 | 4 | 18 | 36 | 77 |
| 33 | P5P-8,8 | 5 | 8 | 25 | >99 |
| 34 | P5P-10,10 | 5 | 10 | 25 | 96 |
| 35 | P5P-11,11 | 5 | 11 | 25 | 94 |
| 36 | P5P-12,12 | 5 | 12 | 25 | 96 |
| 37 | P5P-13,13 | 5 | 13 | 25 | 95 |
| 38 | P5P-14,14 | 5 | 14 | 25 | 92 |
| 39 | P5P-16,16 | 5 | 16 | 25 | 80 |
| 40 | P5P-18,18 | 5 | 16 | 27 | 83 |
| 41 | P6P-8,8 | 6 | 8 | 24 | 94 |
| 42 | P6P-10,10 | 6 | 10 | 24 | 94 |
| 43 | P6P-11,11 | 6 | 11 | 24 | 88 |
| 44 | P6P-12,12 | 6 | 12 | 24 | 87 |
| 45 | P6P-13,13 | 6 | 13 | 24 | 77 |
| 46 | P6P-14,14 | 6 | 14 | 25 | 85 |
| 47 | P6P-16,16 | 6 | 16 | 25 | 83 |
| 48 | P6P-18,18 | 6 | 18 | 25 | 86 |

Next, analogous structures were prepared by the varying alkyl linker up to 6 carbons in length. Application of the same techniques were met with similar success, albeit reactions combining longer hydrocarbon tail lengths and shorter alkyl linkers required extended reaction times for completion. The prepared bisQPCs were classified as PmP-n,n to reflect the alkyl linker length (m) and hydrocarbon tail length (n) and were obtained in yields ≥77%.

It was hypothesized that the synthesis of trisQPCs, could be derived from bis(2-diphenylphosphinoethyl)phenylphosphine in a similar approach as the bisQPCs. Tris-alkylation was possible by treating the starting material with 4.0 equivalents of bromoalkane in acetonitrile at reflux for 96 h (Table 3). To reduce the reaction time, transitioning to more vigorous conditions (DMF at ˜115° C.) proved to be highly successful, generating eight corresponding tris-cationic phosphonium salts in no longer than 24 hours. Due to their structural similarity with the bisQPCs, the trisQPCs were referred to as P2P2P-n,n,n to indicate the ethylene-phosphorus backbone and the hydrocarbon tail length (n).

### Preparation of hexane-1,6-diylbis(decyldiphenylphosphonium), P6P-10,10

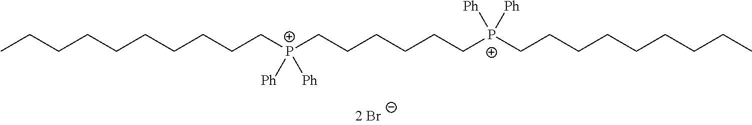 [SMILES: CCCCCCCCCC[P+](CCCCCC[P+](CCCCCCCCC)(c1ccccc1)c1ccccc1)(c1ccccc1)c1ccccc1]

To 1,6-bis(diphenylphosphino)hexane (0.455 g, 1.00 mmol) was added 1-bromodecane (0.553 g, 2.50 mmol) and acetonitrile (3 mL). The solution was heated to reflux and stirred for 24 hours. After cooling to room temperature, the contents of the reaction flask were concentrated using rotary evaporation. The resulting oil was triturated with 1:1 ether:hexanes (25 mL) and cooled at −25° C. overnight. The trituration solvent was discarded, and the resulting precipitate was dissolved in dichloromethane (6 mL). The solution was transferred to a clean vial and concentrated using rotary evaporation to afford P6P-10,10 as a white crystalline powder (0.842 g, 93.8%); mp=75.5-76.3° C.; ¹H NMR (500 MHz, chloroform-d) δ 7.79 (dd, J=12.0, 8.0 Hz, 8H), 7.60 (t, J=7.3 Hz, 4H), 7.54 (td, J=7.5, 2.0 Hz, 8H), 3.18-3.07 (m, 8H), 1.45 (br s, 4H), 1.34 (br s 12H), 1.09-1.02 (m, 24H), 0.68 (t, J=7.0 Hz, 6H); ₁₃C NMR (125.8 MHz, chloroform-d) δ 134.6, 133.2 (d, J=9.7 Hz), 130.3 (d, J=10.8 Hz), 117.9 (d, J=82.1 Hz), 31.8, 30.4 (d, J=15.7 Hz), 29.4, 29.21, 29.18, 29.0, 28.5 (d, J=15.7 Hz), 22.6, 22.2, 21.9 (d, J=49.6 Hz), 21.5 (d, J=41.1 Hz), 21.3 (d, J=7.2 Hz), 14.1; ₃₁P NMR (202.5 MHz, chloroform-d) δ 28.60. HRMS (ESI+): Found 368.2633, C₅₀H₇₄P₂ [M−2Br-]₂₊ requires 368.2628.

TABLE 3
Preparation of trisQPCs.

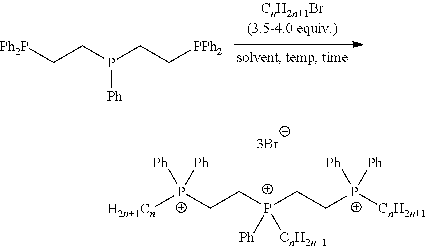

| | | | | temp (° C.), | yield |
|---|---|---|---|---|---|
| compd. | name | n | solvent | time (h) | (%) |
| 49 | P2P2P-8,8,8 | 8 | DMF | 115, 21 | 97 |
| 50 | P2P2P-10,10,10 | 10 | DMF | 120, 24 | 98 |
| 51 | P2P2P-11,11,11 | 11 | CH₃CN | 80, 96 | 86 |
| 52 | P2P2P-12,12,12 | 12 | CH₃CN | 80, 96 | 84 |
| 53 | P2P2P-13,13,13 | 13 | CH₃CN | 80, 96 | 89 |
| 54 | P2P2P-14,14,14 | 14 | DMF | 120, 24 | 90 |
| 55 | P2P2P-16,16,16 | 16 | DMF | 115, 23 | 73 |
| 56 | P2P2P-18,18,18 | 18 | DMF | 110, 24 | 91 |

Experiments were performed to determine whether access to tetra-cationic QPCs may also be possible. Tetra-alkylation of tris[2-(diphenylphosphino)ethyl]phosphine, containing three diphenylphosphine moieties as well as a trialkylphosphine was attained through a one-step synthesis under conditions nearly identical to those of the P2P2P series (Table 4). Employing this approach, tetraQPCs were isolated in high yields and designated as 4P-n,n,n,n to represent the tetraphosphonium core and respective hydrocarbon tail lengths (n).

TABLE 4
Preparation of tetraQPCs

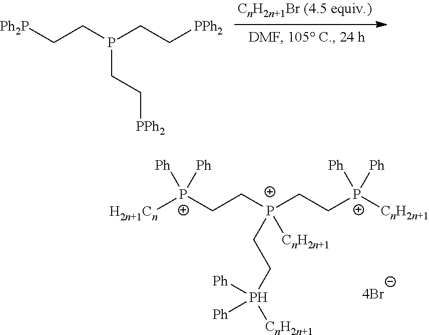

| compd. | name | n | yield (%) |
|---|---|---|---|
| 57 | 4P-10,10,10,10 | 10 | 95 |
| 58 | 4P-11,11,11,11 | 11 | 80 |
| 59 | 4P-12,12,12,12 | 12 | 89 |

### Evaluation of Bioactivity and Toxicity of Quaternary Phosphonium Compounds

Experiments were performed to determine the bioactivities of monoQPCs, bisQPCs, trisQPCs, and tetraQPCs. QPCs bearing long hydrocarbon tails displayed poor water solubility. To mitigate solubility issues, a 2.5% DMSO carrier concentration was used to solubilize the compounds at the highest test concentration (250 μM) and was serially diluted across the remaining test concentrations. The antimicrobial activity, as well as toxicity, were assessed for the QPCs, using red blood cell (RBC) lysis as a proxy for the latter. For comparison to commercially employed QACs, benzalkonium chloride (BAC; 70% benzyldimethyldodecylammonium chloride and 30% benzyldimethyltetradecylammonium chloride) and cetylpyridinium chloride (CPC) were also included in the assays. Furthermore, 2Pyr-11,11,[4] a pyridinium-based bis-cationic QAC displaying best-in-class potency was also included for comparison.

MIC values against a panel of six bacterial strains [community-acquired methicillin-resistant S. aureus (CA-MRSA; USA 300-0114), hospital-acquired methicillin-resistant S. aureus (HA-MRSA; ATCC 33591), methicillin-susceptible S. aureus (SH1000), Enterococcus faecalis (OG1RF), Escherichia coli (MC4100), and Pseudomonas aeruginosa (PAO1)] along with the RBC lysis (presented as Lysis20), is presented in Table 5.

TABLE 5
Antimicrobial activity and cytotoxicity, measured as MIC and Lysis₂₀,

| | | Minimum Inhibitory Concentration (μM) | | | | | | |
|---|---|---|---|---|---|---|---|---|
| | | | CA- | HA- | | | | Lys₂₀ |
| | Compound | MSSA | MRSA | MRSA | E. faecalis | E. coli | Pa^{b} | (μM)^{a} |
| QACs | BAC | 4 | 4 | 8 | 250 | 63 | 250 | 16 |
| | CPC | 1 | 1 | 2 | 250 | 32 | 250 | 4 |
| | 2Pyr-11,11 | 1 | 2 | 2 | 8 | 2 | 16 | 4 |
| monoQPCs | TPP-8 | 4 | 4 | 125 | >250 | >250 | >250 | 63 |
| | TPP-10 | 1 | 2 | 8 | 250 | 250 | 250 | 16 |
| | TPP-11 | 1 | 1 | 4 | 125 | 125 | 250 | 16 |
| | TPP-12 | 0.5 | 1 | 2 | 63 | 250 | 63 | 8 |
| | TPP-13 | 1 | 1 | 2 | 16 | 4 | 32 | 4 |
| | TPP-14 | 2 | 2 | 2 | 8 | 16 | 16 | 4 |
| | TPP-16 | 2 | 2 | 4 | 4 | 16 | 16 | 4 |
| | TPP-18 | 4 | 8 | 8 | 16 | 125 | 125 | 4 |
| bisQPCs | P2P-8,8 | 1 | 0.5 | 8 | 250 | >250 | 250 | 63 |
| | P2P-10,10 | 1 | 1 | 2 | 8 | 16 | 8 | 4 |
| | P2P-11,11 | 1 | 1 | 2 | 8 | 32 | 16 | 4 |
| | P2P-12,12 | 1 | 2 | 8 | 32 | 63 | 63 | 4 |
| | P3P-8,8 | 1 | 1 | 4 | 250 | >250 | 250 | 63 |
| | P3P-10,10 | 2 | 1 | 2 | 4 | 63 | 4 | 2 |
| | P3P-11,11 | 1 | 1 | 16 | 4 | 8 | 16 | 4 |
| | P3P-12,12 | 1 | 1 | 4 | 32 | 125 | 125 | 4 |
| | P3P-13,13 | 2 | 2 | 4 | 63 | 63 | 63 | 4 |
| | P3P-14,14 | 2 | 2 | 8 | 125 | 63 | >250 | 2 |
| | P4P-8,8 | 1 | 1 | 4 | 125 | 250 | 125 | 63 |
| | P4P-10,10 | 1 | 2 | 2 | 4 | 16 | 8 | 2 |
| | P4P-11,11 | 1 | 2 | 2 | 4 | 16 | 16 | 2 |
| | P4P-12,12 | 2 | 2 | 4 | 16 | 63 | >250 | 1 |
| | P4P-13,13 | 2 | 2 | 8 | 125 | 125 | >250 | 2 |
| | P4P-14,14 | 8 | 4 | 4 | 63 | 125 | >250 | 1 |
| | P5P-8,8 | 0.5 | 1 | 2 | 125 | 250 | 125 | 32 |
| | P5P-10,10 | 1 | 1 | 4 | 2 | 16 | 4 | 2 |
| | P5P-11,11 | 2 | 2 | 4 | 2 | 8 | 16 | 2 |
| | P5P-12,12 | 2 | 2 | 4 | 16 | 32 | 63 | 2 |
| | P5P-13,13 | 4 | 4 | 4 | 63 | 125 | 250 | 2 |
| | P5P-14,14 | 4 | 8 | 8 | 63 | 125 | >250 | 1 |
| | P5P-16,16 | 8 | 16 | 16 | 125 | 125 | 250 | 1 |
| | P6P-8,8 | 0.5 | 0.5 | 2 | 63 | 125 | 63 | 16 |
| | P6P-10,10 | 1 | 1 | 2 | 2 | 8 | 4 | 2 |
| | P6P-11,11 | 2 | 2 | 4 | 2 | 16 | 16 | 2 |
| | P6P-12,12 | 2 | 2 | 2 | 16 | 32 | 32 | 2 |
| | P6P-13,13 | 4 | 2 | 4 | 32 | 63 | >250 | 2 |
| | P6P-14,14 | 4 | 4 | 8 | 63 | 63 | 250 | 2 |
| trisQPCs | P2P2P-8,8,8 | 2 | 1 | 2 | 250 | 250 | >250 | 16 |
| | P2P2P-10,10,10 | 2 | 2 | 8 | 250 | 32 | >250 | 2 |
| | P2P2P-11,11,11 | 2 | 4 | 4 | 63 | 63 | >250 | 1 |
| | P2P2P-12,12,12 | 2 | 4 | 8 | 125 | 125 | >250 | 1 |
| | P2P2P-13,13,13 | 2 | 4 | 16 | 125 | 125 | >250 | 2 |
| | P2P2P-14,14,14 | 16 | 16 | 32 | 250 | 250 | >250 | 1 |
| tetraQPCs | 4P-10,10,10,10 | 16 | 32 | 32 | 250 | 250 | >250 | 8 |
| | 4P-11,11,11,11 | 16 | 32 | 125 | 125 | 125 | >250 | 4 |
| | 4P-12,12,12,12 | 16 | 16 | 63 | 250 | 250 | >250 | 4 |

Inspection of the bioactivity profiles of the monoQPCs indicated trends that were unique from the multi-cationic species examined herein. Optimal activity against MSSA, CA-MRSA, and HA-MRSA was observed for compounds bearing 11-13-carbon tail lengths (MIC=0.5, 1, and 2 M, respectively). Additionally, TPP-13 displayed the greatest potency amongst the monoQPCs against the Gram-negative strains, with a ˜63-fold increase in activity compared to its 12-carbon analog against E. coli (MIC=4 μM). Interestingly, at longer chain lengths, activity against E. faecalis and P. aeruginosa continued to increase, with TPP-16 resulting in ˜63-fold and ˜16-fold increases in activity (MIC=4 and 16 μM, respectively) compared to the commercially available QACs, BAC and CPC.

In comparison to the monoQPCs, the bisQPCs followed a slightly different pattern in bioactivity, with compounds bearing shorter tail lengths displaying optimal activities for all strains.

Against MSSA and CA-MRSA, compounds with 8-carbon tail lengths displayed the best activities, with P2P-8,8, P5P-8,8, and P6P-8,8 each reporting sub-micromolar activity (MIC=0.5 μM) for one of the two strains. In contrast, against E. faecalis, E. coli, and P. aeruginosa, shorter alkyl tail lengths of 10 and 11 carbons resulted in 4-fold and greater enhancements in activity compared to their 8-carbon counterparts. However, as the hydrocarbon tail length increased beyond 11 carbons, activity across all strains began to decline, with this trend being most evident against E. faecalis, E. coli, and P. aeruginosa. These findings contrast our previous investigations of multiQACs, in which growing chain lengths displayed an improvement in activity.

Further examining the bisQPCs, the effect of varying the alkyl linker separating the two quaternized phosphines was also a subject of interest. While this modification led to nominal changes in activity for five of the strains, increasing the alkyl linker length did lead to a moderate increase in optimal activity against E. faecalis. Notably, P6P-10,10, bearing a 6-carbon linker, displayed the greatest broad-spectrum activity of all the prepared QPCs. Furthermore, P6P-10,10 also compared favorably to the best-in-class bis-cationic QAC, 2Pyr-11,11.

Analysis of the multiQPCs unfortunately revealed no increase in bioactivity corresponding to the additional phosphonium residues. Rather, tris-cationic multiQPCs unveiled a similar trend to that of the bisQPCs, with shorter alkyl tail lengths (8-11 carbons) exhibiting greater potencies. In particular, P2P2P-8,8,8 illustrated the best activity against the MSSA, CA-MRSA, and HA-MRSA strains (MIC=2, 1, and 2 μM, respectively). However, in general, increasing the number of phosphonium atoms led to an overall decrease in antimicrobial activity for both tris- and tetra-cationic QPCs compared to their bis-cationic counterparts.

Red blood cell lysis (measured as Lysis20), serving as an approximation for cytotoxicity, appeared to parallel alkyl chain length for mono- and bisQPCs. For mono-, bis-, and trisQPCs, 8-carbon species consistently displayed the lowest hemolytic activity, with no compound reporting toxicity greater than BAC (Lysis₂₀≥16 μM). However, increasing the chain length from 8 to 10 carbons had a profound impact on toxicity, leading to at least an 8-fold increase in hemolytic activity for bis- and trisQPCs. For compounds with alkyl chains above 10 carbons, hemolytic activity continued to gradually increase. Additionally, the length of the alkyl linker had a minor impact on the toxicity of bisQPCs, with longer spacers leading to increased hemolytic activity. These results parallel previous studies on antimicrobial and anticancer amphiphiles in which increasing hydrocarbon tail length correlates with increasing cytotoxicity due to membrane disruption.

### Implication of SMR Family Transporters in QPC Resistance

Upon comparison of the QPC bioactivities between strains, a pattern of resistance for HA-MRSA emerged amongst the S. aureus strains. Specifically, mono- and bisQPCs possessing short to moderate hydrocarbon tail lengths (8-12 carbons) displayed significantly higher MICs against the HA-MRSA strain compared the MSSA and CA-MRSA strains. Experiments were performed to determine whether this observed resistance may be the result of the distinct presence of an efflux pump in HA-MRSA. Subsequent genomic analysis comparing the HA-MRSA, CA-MRSA, and MSSA strain resistomes indeed unveiled the unique presence of the qacC gene in HA-MRSA. The qacC gene encodes the Small Multidrug Resistance (SMR) family transporter protein, QacC which is implicated in the efflux of quaternary ammonium compounds, in addition to methyltriphenylphosphonium and tetraphenylphosphonium. Notably, the Gram-negative QacC homolog, EmrE, was also identified in both the E. coli and P. aeruginosa strain genomes; furthermore, QacE, a closely related SMR family efflux pump was identified in the E. faecalis strain genome (FIG. 2). Taken together, the presence of these Small Multidrug Pump (SMP) type SMR transporters may be responsible for the refractory nature of these strains to treatment with QAC and QPC disinfectants.

Experiments reported herein indicate a relationship between alkyl chain lengths and number of cationic residues with antimicrobial efficacy and hemolytic activity for QPCs. Specifically, both monoQPCs with moderate hydrocarbon tail lengths (11-13 carbons) and bisQPCs with shorter tail lengths (8-11 carbons) demonstrated promising antimicrobial activities against a panel of six Gram-positive and Gram-negative bacteria. Notably, P6P-10,10 emerged as an effective broad-spectrum antimicrobial, exhibiting comparable hemolytic activity and ≥4-fold increases in activity against Gram-negative species compared to commercial QACs, BAC and CPC. Furthermore, this bisQPC was able to evade HAMRSA QPC-resistance potentially conferred by the presence of qacC, a gene encoding multidrug efflux pump, QacC. The recent spread of the qacC gene underscores the need for next-generation disinfectants, such as P6P-10,10, that overcome mounting resistance.

Quaternary Phosphonium Compounds (QPC) as Disinfectants Against Highly Resistant Acinetobacter baumannii Clinical Isolates

Acinetobacter baumannii is classified as a highest threat pathogen, urgently necessitating antimicrobials that evade resistance to combat its spread. Quaternary ammonium compounds (QACs, e.g., 2-Pyr11,11) have afforded a valuable first line-of-defense against antimicrobial resistant pathogens as broad-spectrum amphiphilic disinfectant molecules. However, QAC resistance can occur.

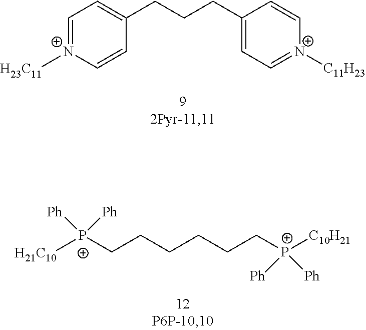

Quaternary ammonium and phosphonium compounds were test against a panel of resistant A. baumannii clinical isolates. QPC (P6P-10,10) displayed improved activities compared to other QACs.

Table 6. Comparison of the minimum biofilm eradication concentration (MBEC) for leading commercial disinfectants, BAC and DDAC, compared to 2Pyr-11,11 and P6P-10,10. Compounds were evaluated against A. baumannii strain ATCC 19606 and pan-resistant clinical isolate, MRSN 17493.

MBEC (μM)

| | ATCC 19606 | MRSN 17493 |
|---|---|---|
| BAC | 2000 | 2000 |
| DDAC | 1000 | 500 |
| BAC/DDAC | 1000 | 1000 |
| 2Pyr-11,11 | 1000 | 500 |
| P6P-10,10 | 500 | 63 |

P6P-10,10 maintained efficacy against the A. baumannii strain with an IC₉₀ of 3 μM and minimum biofilm eradication concentration of 63 μM. QAC-resistant A. baumannii mutants were generated and observed the development of QAC cross-resistance. In contrast, neither disinfectant resistance nor cross-resistance was observed in A. baumannii under P6P-10,10 treatment.

1. A quaternary phosphonium compound having Formula I, 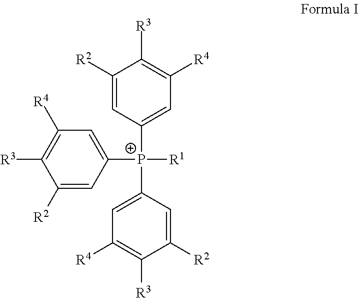
wherein
R¹ is a lipid and
R², R³, and R⁴ are each individually and independently selected from hydrogen, alkyl, alkoxy, or halogen, wherein R², R³, and R⁴ are optionally substituted.

2. The compound of claim 1 wherein the R¹ lipid is a saturated or unsaturated hydrocarbon with eight or greater carbons.

3. The compound of claim 1 which is selected from:
octyltriphenylphosphonium,
decyltriphenylphosphonium,
triphenyl(undecyl)phosphonium,
dodecyltriphenylphosphonium,
triphenyl(tridecyl)phosphonium,
triphenyl(tetradecyl)phosphonium,
hexadecyltriphenylphosphonium, and
octadecyltriphenylphosphonium.

4. A quaternary phosphonium compound having Formula II, 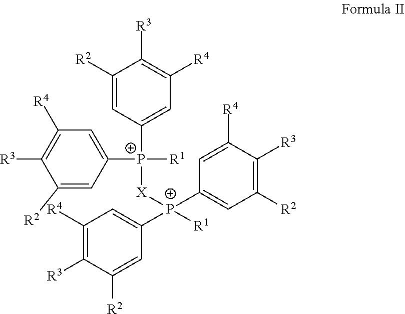
wherein,
R¹ is a lipid;
X is a linking group; and
R², R³, and R⁴ are each individually and independently selected from hydrogen, alkyl, alkoxy, or halogen, wherein R², R³, and R⁴ are optionally substituted.

5. The compound of claim 4, wherein the R¹ lipid is a saturated or unsaturated hydrocarbon with eight or greater carbons.

6. The compound of claim 4, wherein the X linking group is a saturated or unsaturated hydrocarbon with two or greater carbons.

7. The compound of claim 4 which is selected from:
ethane-1,2-diylbis(octyldiphenylphosphonium)
ethane-1,2-diylbis(decyldiphenylphosphonium),
ethane-1,2-diylbis(undecyldiphenylphosphonium),
ethane-1,2-diylbis(dodecyldiphenylphosphonium),
ethane-1,2-diylbis(tridecyldiphenylphosphonium),
ethane-1,2-diylbis(tetradecyldiphenylphosphonium),
ethane-1,2-diylbis(hexadecyldiphenylphosphonium),
ethane-1,2-diylbis(octadecyldiphenylphosphonium),
propane-1,3-diylbis(octyldiphenylphosphonium),
propane-1,3-diylbis(decyldiphenylphosphonium),
propane-1,3-diylbis(undecyldiphenylphosphonium),
propane-1,3-diylbis(dodecyldiphenylphosphonium),
propane-1,3-diylbis(tridecyldiphenylphosphonium),
propane-1,3-diylbis(tetradecyldiphenylphosphonium),
propane-1,3-diylbis(hexadecyldiphenylphosphonium),
propane-1,3-diylbis(octadecyldiphenylphosphonium),
butane-1,4-diylbis(octyldiphenylphosphonium),
butane-1,4-diylbis(decyldiphenylphosphonium),
butane-1,4-diylbis(undecyldiphenylphosphonium),
butane-1,4-diylbis(dodecyldiphenylphosphonium),
butane-1,4-diylbis(tridecyldiphenylphosphonium),
butane-1,4-diylbis(tetradecyldiphenylphosphonium),
butane-1,4-diylbis(hexadecyldiphenylphosphonium),
butane-1,4-diylbis(octadecyldiphenylphosphonium),
pentane-1,5-diylbis(octyldiphenylphosphonium),
pentane-1,5-diylbis(decyldiphenylphosphonium),
pentane-1,5-diylbis(undecyldiphenylphosphonium),
pentane-1,5-diylbis(dodecyldiphenylphosphonium),
pentane-1,5-diylbis(tridecyldiphenylphosphonium),
pentane-1,5-diylbis(tetradecyldiphenylphosphonium),
pentane-1,5-diylbis(hexadecyldiphenylphosphonium),
pentane-1,5-diylbis(octadecyldiphenylphosphonium),
hexane-1,6-diylbis(octyldiphenylphosphonium),
hexane-1,6-diylbis(decyldiphenylphosphonium),
hexane-1,6-diylbis(undecyldiphenylphosphonium),
hexane-1,6-diylbis(dodecyldiphenylphosphonium),
hexane-1,6-diylbis(tridecyldiphenylphosphonium),
hexane-1,6-diylbis(tetradecyldiphenylphosphonium),
hexane-1,6-diylbis(hexadecyldiphenylphosphonium), and
hexane-1,6-diylbis(octadecyldiphenylphosphonium).

8. A quaternary phosphonium compound having Formula III, 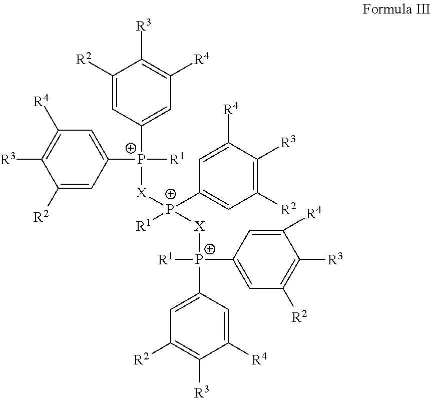
wherein,
R¹ is a lipid;
X is a linking group; and
R², R³, and R⁴ are each individually and independently selected from hydrogen, alkyl, alkoxy, or halogen, wherein R², R³, and R⁴ are optionally substituted.

9. The compound of claim 8, wherein the R¹ lipid is a saturated or unsaturated hydrocarbon with eight or greater carbons.

10. The compound of claim 8, wherein the X linking group is a saturated or unsaturated hydrocarbon with two or greater carbons.

11. The compound of claim 8 which is selected from:
((octyl(phenyl)phosphoniodiyl)bis(ethane-2,1-diyl))bis(octyldiphenylphosphonium),
((decyl(phenyl)phosphoniodiyl)bis(ethane-2,1-diyl))bis(decyldiphenylphosphonium),
((phenyl(undecyl)phosphoniodiyl)bis(ethane-2,1-diyl))bis(diphenyl(undecyl)phosphonium),
((dodecyl(phenyl)phosphoniodiyl)bis(ethane-2,1-diyl))bis(dodecyldiphenylphosphonium),
((phenyl(tridecyl)phosphoniodiyl)bis(ethane-2,1-diyl))bis(diphenyl(tridecyl)phosphonium),
((phenyl(tetradecyl)phosphoniodiyl)bis(ethane-2,1-diyl))bis(diphenyl(tetradecyl)phosphonium),
((hexadecyl(phenyl)phosphoniodiyl)bis(ethane-2,1-diyl))bis(hexadecyldiphenylphosphonium), and
((octadecyl(phenyl)phosphoniodiyl)bis(ethane-2,1-diyl))bis(octadecyldiphenylphosphonium).

12. A quaternary phosphonium compound having Formula IV, 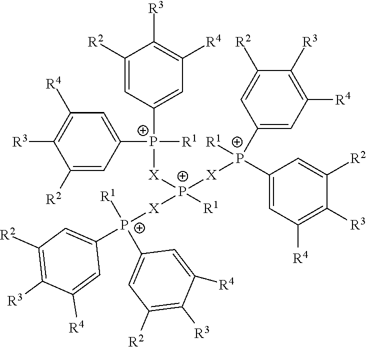
wherein,
R¹ is a lipid;
X is a linking group; and
R², R³, and R⁴ are each individually and independently selected from hydrogen, alkyl, alkoxy, or halogen, wherein R², R³, and R⁴ are optionally substituted.

13. The compound of claim 12, wherein the R¹ lipid is a saturated or unsaturated hydrocarbon with eight or greater carbons.

14. The compound of claim 12, wherein the X linking group is a saturated or unsaturated hydrocarbon with two or greater carbons.

15. The compound of claim 12 which is selected from:
((decylphosphoniotriyl)tris(ethane-2,1-diyl))tris(decyldiphenylphosphonium),
((undecylphosphoniotriyl)tris(ethane-2,1-diyl))tris(diphenyl(undecyl)phosphonium), and
((dodecylphosphoniotriyl)tris(ethane-2,1-diyl))tris(dodecyldiphenylphosphonium).

16. A pharmaceutical formulation comprising a quaternary phosphonium compound as provided in claim 1 and a pharmaceutically acceptable excipient.

17. The pharmaceutical formulation of claim 16 in the form of a particle, bead, tablet, capsule, or pill.

18. The pharmaceutical formulation of claim 16 in the form of a lotion, liquid, or gel.

19. A medical device coated with a quaternary phosphonium compound as provided in claim 1.

20. A method of treating or preventing a microbial infection comprising administering to a subject in need thereof an effective amount of a quaternary phosphonium compound as provided in claim 1.

21. The method of claim 20, wherein the microbial infection is a bacterial, fungal, or viral infection.
